(19) United States
(12) Patent Application Publication
(10) Pub. No.: US 2025/0095793 A1
(43) Pub. Date: Mar. 20, 2025

(54) METHODS AND SYSTEMS FOR STUDYING MOLECULES AND PROPERTIES THEREOF

(71) Applicant: GOOD CHEMISTRY INC.

(72) Inventors: Takeshi YAMAZAKI, Vancouver (CA); Kevin RYCZKO, Gatineau (CA); Arman ZARIBAFIYAN, Vancouver (CA); Nima ALIDOUST, Redwood City, CA (US)

(21) Appl. No.: 18/814,910

(22) Filed: Aug. 26, 2024

Related U.S. Application Data
(63) Continuation of application No. PCT/IB2023/051819, filed on Feb. 27, 2023.
(60) Provisional application No. 63/314,971, filed on Feb. 28, 2022.

(51) Int. Cl. G16C 20/30; G16B 15/20; G16B 15/30; G16C 20/70

(52) U.S. Cl. CPC G16C 20/30; G16B 15/20; G16B 15/30; G16C 20/70

(57) ABSTRACT

Provided herein is a method for studying molecule and properties thereof, the method comprising: a) obtaining a request comprising an indication of at least one property of a molecule and a corresponding task; b) performing inference on at least one machine learning (ML) model using said indication, the at least one machine learning (ML) model configured to mimic result of said task to generate an inference outcome; c) performing inference reliability test on the inference outcome; i) obtaining task result using the inference outcome in response to a satisfactory inference reliability test; ii) performing said task using said indication to obtain task result in response to a nonsatisfactory inference reliability test; d) outputting said task result.

### CROSS-REFERENCE TO RELATE APPLICATION

This application is a continuation of International Application No. PCT/IB2023/051819 filed Feb. 27, 2023, which claims the benefit of U.S. Provisional Patent Application No. 63/314,971 filed Feb. 28, 2022, the disclosure of which is incorporated herein by reference in its entirety.

### BACKGROUND

Computational chemistry has become an established tool for the molecular and material discovery process in many areas of industry. Computational chemistry can provide accurate prediction of chemical phenomena and examination of molecular properties that may be inaccessible solely from the experiment and/or requires significant labor. There are several computational chemistry platforms to facilitate accurate prediction of chemical behavior of molecules and materials. Some examples of such platforms include Gaussian™ (https://gaussian.com), Q-Chem™ (https://www.q-chem.com), and Schrodinger™ (https://www.schrodinger.com). For these platforms, the improvement of the computational efficiency is one of the most essential development activities to help accelerate the material discovery process, and the platform developers have been putting an enormous effort to improve the efficiency without sacrificing the accuracy level of the computational prediction. The example can be found in the Efficiency Improvements section in https://gaussian.com/gdiffs/.

However, since the efficiency improvement of these platforms is performed based on their development roadmap, users need to be endured the provided level of efficiency in computational approaches that satisfy their accuracy needs until the new package with the efficiency improvement is released and/or users need to choose less accurate but more efficient computational approaches for their efficiency needs. This causes an undesired delay in the users' research progress and/or a failure in accurate prediction due to the usage of less accurate approaches. There are several open-source packages for computational chemistry, such as PySCF(https://pyscf.org) and Psi4 (https://psicode.org), in which the users can directly contribute to the development of computational chemistry methods in the package and hence the level of their efficiency. However, the efficiency improvement of the methods requires significant labor and deep knowledge and experience in the computational chemistry methods.

### SUMMARY

Recognized herein is the need for improved methods and systems that will overcome at least one of the above-identified drawbacks. The present disclosure provides methods and systems for studying molecule and properties thereof. The present disclosure may improve upon existing methods and systems in at least some aspects.

An advantage of one or more embodiments of the methods and systems disclosed herein may be that they provide a computational chemistry platform that improves its computational efficiency by having users run computational chemistry calculations/pipeline on the platform.

An advantage of one or more embodiments of the methods and systems disclosed herein may be that they provide a computational chemistry platform that efficiency improvement happens more frequently as users run more frequently computational chemistry calculations/pipeline on the platform.

An advantage of one or more embodiments of the methods and systems disclosed herein may be that they provide a computational chemistry platform that becomes more efficient as users run more computational chemistry calculations/pipeline on the platform.

An advantage of one or more embodiments of the methods and systems disclosed herein may be that they provide a computational chemistry platform the efficiency of which can be improved without human labor to improve the efficiency of computational chemistry methods.

An advantage of one or more embodiments of the methods and systems disclosed herein may be that they provide a computational chemistry platform the efficiency of which can be improved without deep knowledge or experience in improving the efficiency of computational chemistry methods.

An advantage of one or more embodiments of the methods and systems disclosed herein may be that they provide a computational chemistry platform the efficiency of which can be improved by conducting laboratory experiments.

Another advantage of one or more embodiments of the methods and systems disclosed herein may be that they provide a framework which may be realized by combining machine learning techniques and the chemistry discovery toolkit that may consist of various computational chemistry techniques, computational discovery pipelines, and also laboratory experiments.

In an aspect, the present disclosure provides a method for studying molecule and properties thereof, the method comprising: a) obtaining a request comprising an indication of at least one property of a molecule and a corresponding task; b) performing inference on at least one machine learning (ML) model using said indication, the at least one machine learning (ML) model configured to mimic result of said task to generate an inference outcome; c) performing inference reliability test on the inference outcome; i) obtaining task result using the inference outcome in response to a satisfactory inference reliability test; ii) performing said task using said indication to obtain task result in response to a nonsatisfactory inference reliability test; d) outputting said task result.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 is a diagram of a system for studying molecule and properties thereof in accordance with some embodiments disclosed herein.

FIG. 2 is a flowchart of a method for studying molecule and properties thereof in accordance with some embodiments disclosed herein.

FIG. 3 is a flowchart of an embodiment of a method for calculating the ground state energy with DFT wB97X/6-31g* level accuracy based on the three-dimensional coordinate of a molecule.

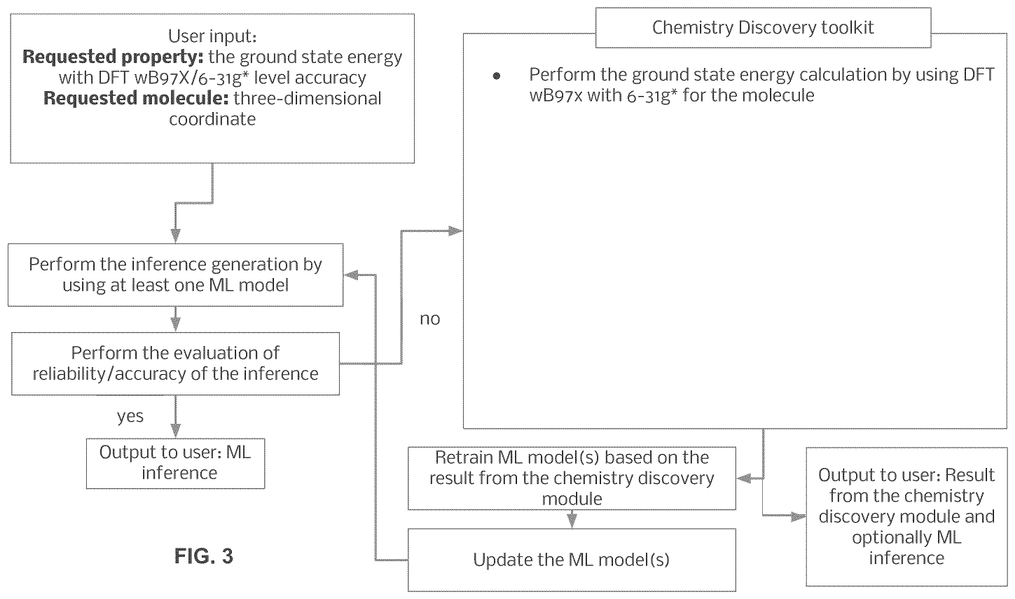

FIG. 4 is a flowchart of an embodiment of a method for calculating the ground state optimized geometry with CCSD(T)/CBS level accuracy based on SMILES.

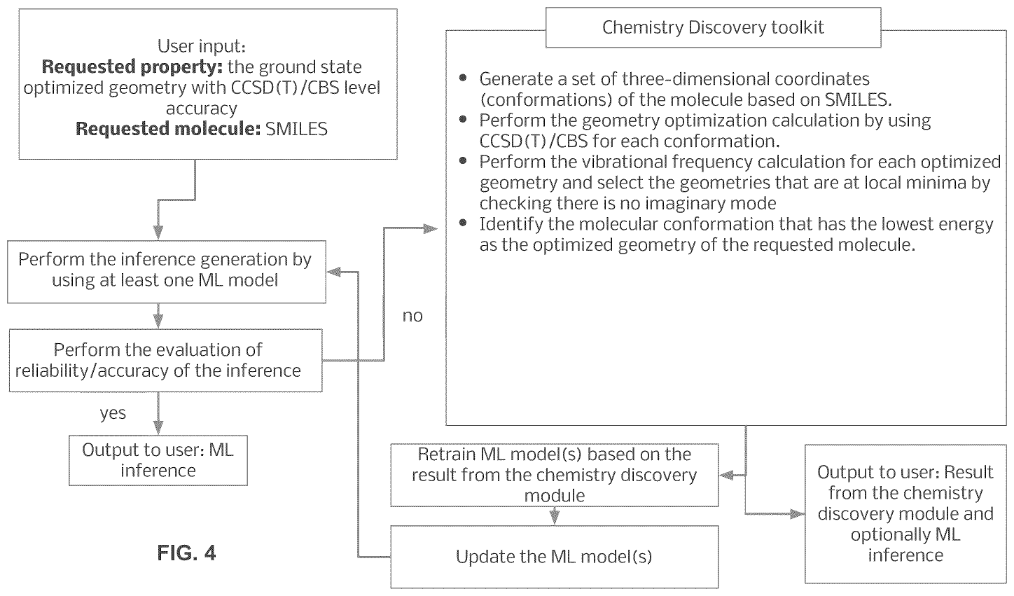

FIG. 5 is a flowchart of an embodiment of a method for obtaining barrier height of a target chemical reaction with DFT B3LYP/cc-pvd based on three-dimensional coordinates of a reactant and product.

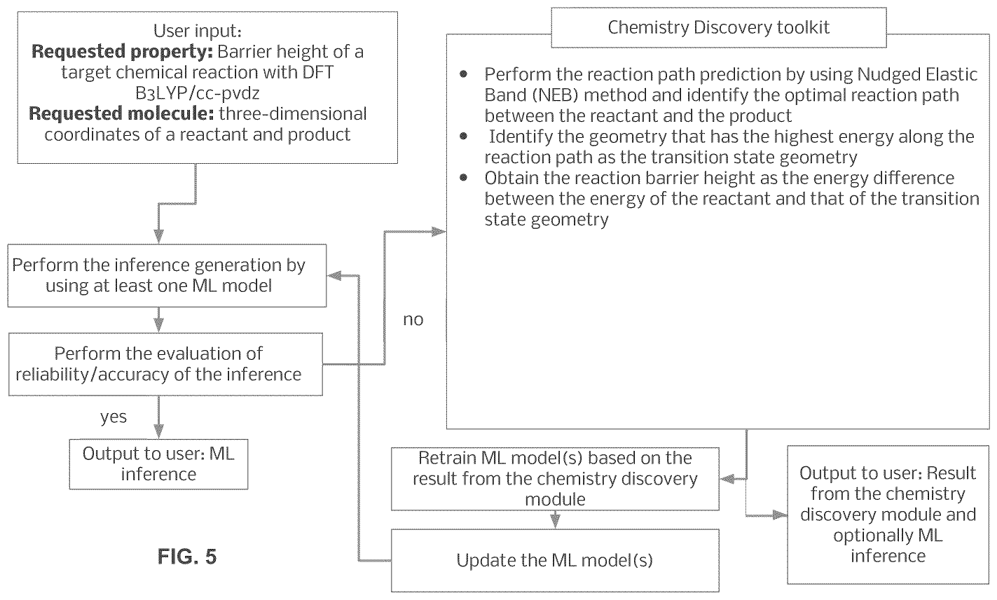

FIG. 6 is a flowchart of an embodiment of a method for calculating UV-Vis spectrum of a target molecule with TDDFT PBE0/6-311+G(d,p) based on the three-dimensional coordinate of a molecule.

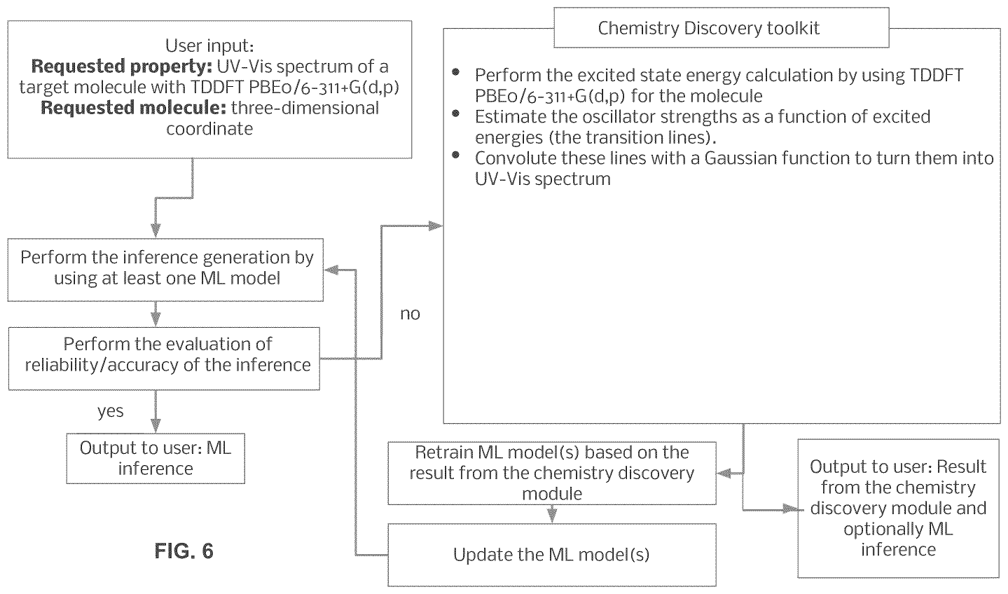

FIG. 7 is a flowchart of an embodiment of a method for obtaining a list of organic molecules that have the strong peaks at 350 nm and 500 nm in UV-Vis spectrum and have the number of heavy atoms less than 15.

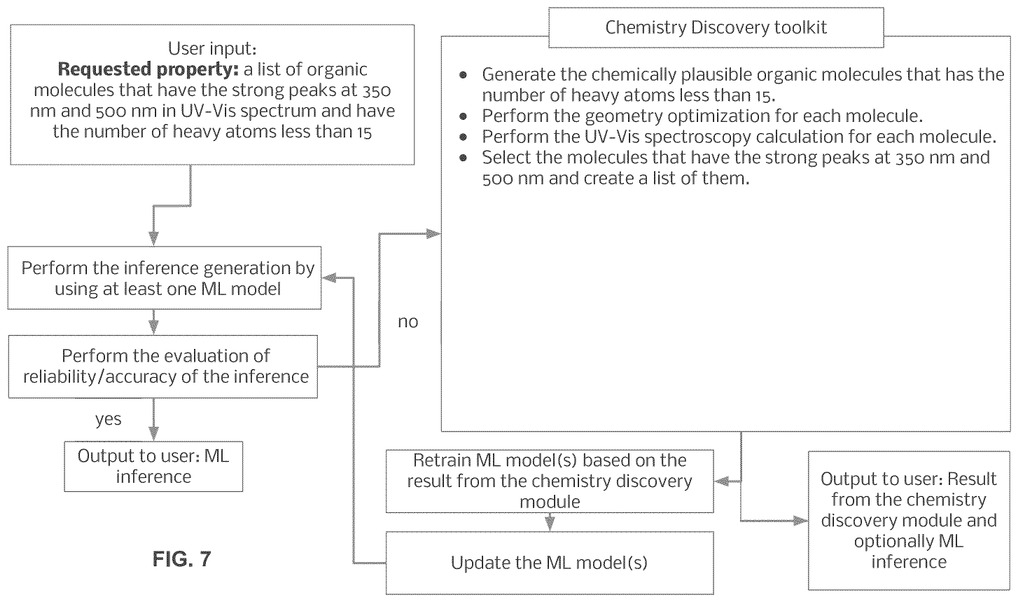

FIG. 8 is a flowchart of an embodiment of a method for estimating residence time of a ligand in the ligand binding site of the target protein based on three-dimensional coordinate of the binding conformation between the ligand and protein.

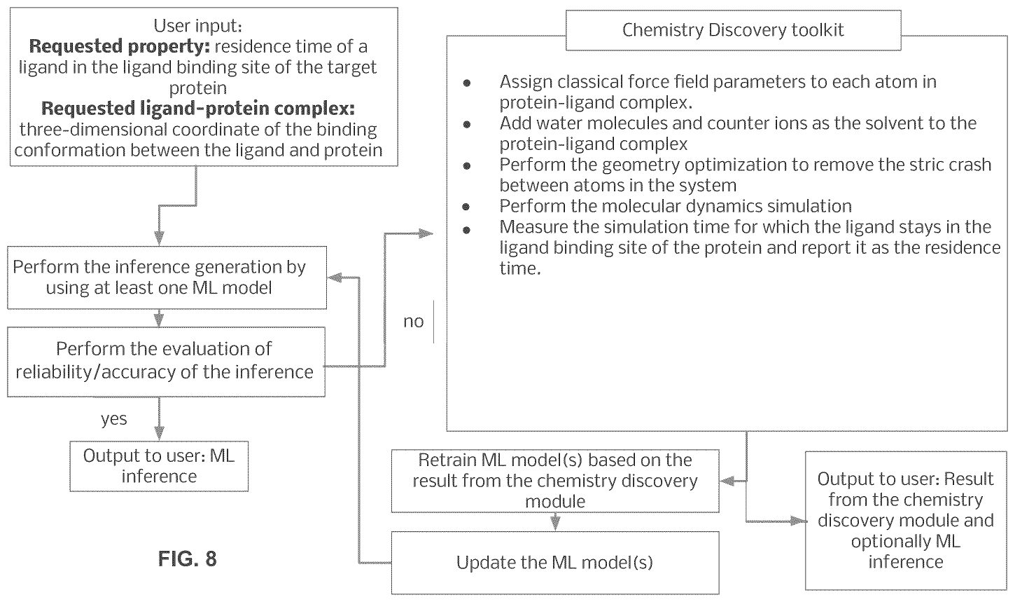

FIG. 9 is a flowchart of an embodiment of a method for identifying the ligand from a ligand database that has the strongest binding free energy with a target protein PDB ID based on SMILES from a ligand library.

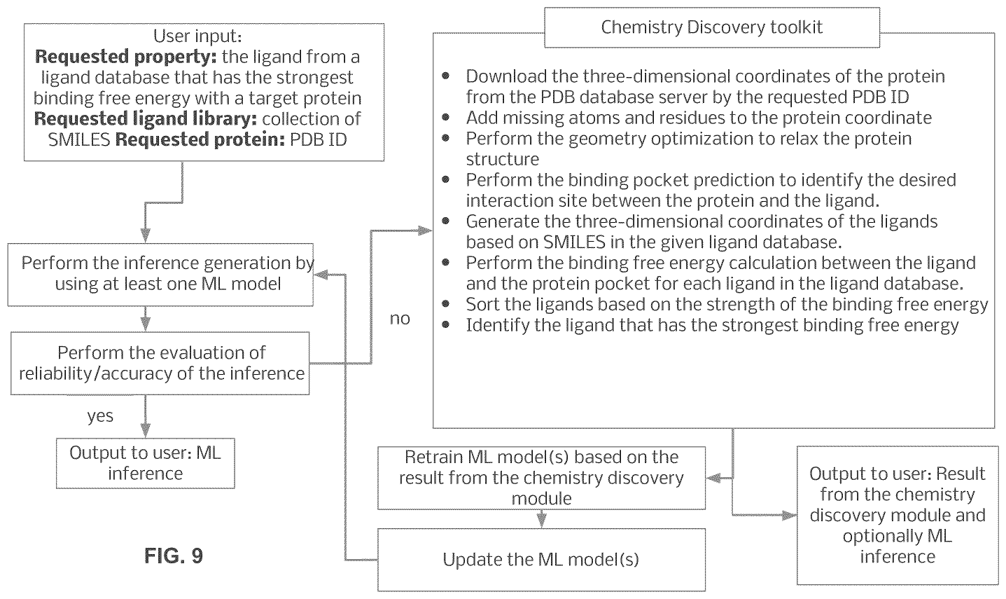

FIG. 10 is a flowchart of an embodiment of a method for identifying self-complementary Janus type motif, based on guanine and cytosine base pairs, that forms a nanotube with the 3.7 nm outer diameter.

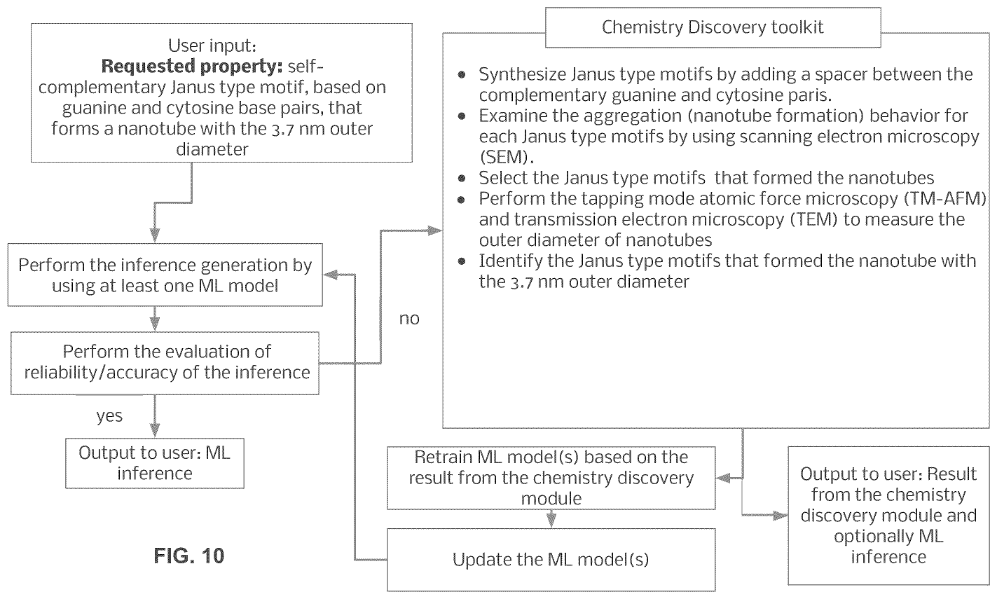

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated.

The term “plurality” means “two or more,” unless expressly specified otherwise.

The term “herein” means “in the present application, including anything which may be incorporated by reference,” unless expressly specified otherwise.

The term “e.g.” and like terms mean “for example,” and thus do not limit the terms or phrases they explain. For example, in a sentence “the computer sends data (e.g., instructions, a data structure) over the Internet,” the term “e.g.” explains that “instructions” are an example of “data” that the computer may send over the Internet, and also explains that “a data structure” is an example of “data” that the computer may send over the Internet. However, both “instructions” and “a data structure” are merely examples of “data,” and other things besides “instructions” and “a data structure” can be “data.”

Where values are described as ranges, the disclosure includes the disclosure of all possible sub-ranges within such ranges, as well as specific numerical values that fall within such ranges irrespective of whether a specific numerical value or specific sub-range is expressly stated.

In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be used, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

As used herein, the term “classical,” as used in the context of computing or computation, generally refers to computation performed using binary values using discrete bits without use of quantum mechanical superposition and quantum mechanical entanglement. A classical computer may be a digital computer, such as a computer employing discrete bits (e.g., 0's and 1's) without use of quantum mechanical superposition and quantum mechanical entanglement.

As used herein, the term “non-classical,” as used in the context of computing or computation, generally refers to any method or system for performing computational procedures outside of the paradigm of classical computing.

As used herein, the term “quantum device” generally refers to any device or system to perform computations using any quantum mechanical phenomenon such as quantum mechanical superposition and quantum mechanical entanglement.

As used herein, the terms “quantum computation,” “quantum procedure,” “quantum operation,” and “quantum computer” generally refer to any method or system for performing computations using quantum mechanical operations (such as unitary transformations or completely positive trace-preserving (CPTP) maps on quantum channels) on a Hilbert space represented by a quantum device.

As used herein, the term “Noisy Intermediate-Scale Quantum device” (NISQ) generally refers to any quantum device which is able to perform tasks which surpass the capabilities of today's classical digital computers.

As used herein, the term “quantum chemistry calculation” generally refers to the calculations to predict the electronic structure and molecular properties using quantum mechanics.

As used herein, the “molecular mechanical calculation” generally refers to the molecular modeling calculations based on classical mechanics.

As used herein, the “computational chemistry calculation” generally refers to a computer simulation to assist in solving chemical problems. Computational chemistry calculation may comprise quantum chemistry calculations. The computational chemistry calculation may further comprise molecular mechanical calculations.

As used herein, the “chemistry discovery toolkit” generally refers to the toolkit to help the design and discovery of new molecules and properties of a molecule. The chemistry discovery toolkit may comprise computational chemistry methods, molecular mechanical methods, and quantum chemistry methods. The chemistry discovery toolkit may further comprise a set of aforementioned methods that are performed in a specific order (pipeline). The chemistry discovery toolkit may further comprise laboratory experiment approaches.

As used herein, the term “machine learning (ML) model” generally refers to a flexible algorithm that has been trained to recognize certain types of patterns. ML algorithms include, but are not limited to, Supervised Learning, Semi-supervised learning, Unsupervised Learning, Transfer Learning, Delta Learning, and Reinforcement Learning.

As used herein, the term “molecule” generally refers to the substance that is made up of one or more atoms. It can include condensed phase systems such as solid and liquid. It can further include materials.

As used herein, the term “property of a molecule” generally refers to the properties including the chemical properties, physical properties, and structural properties of molecules. It can also include pharmacological or biological properties of the molecule.

As used herein, the term “fingerprint” generally refers to a way of uniquely encoding the structure of a molecule.

As used herein, the term “ground state” generally refers to the lowest-energy state of the system. The state can include the electronic energy state, vibrational energy state, and the rotational energy state.

As used herein, the term “excited state” generally refers to the states with energy greater than the ground state. The state can include the electronic energy state, vibrational energy state, and the rotational energy state.

As used herein, the term “molecular orbital” generally refers to a mathematical function describing the location and wave-like behavior of an electron in a molecule.

As used herein, the term “ionization potential” generally refers to the amount of energy required to remove an electron from a neutral atom or molecule.

As used herein, the term “electron affinity” generally refers to the amount of energy released when an electron is attached to a neutral atom or molecule

As used herein, the term “singlet-triplet gap” generally refers to the energy difference between the singlet spin state and the triplet spin state of a system.

As used herein, the term “atomic charge” generally refers to the real numbers describing the distribution of electron density in a molecule.

As used herein, the term “dipole moment” generally refers to the measure of the separation of positive and negative electrical charges within a system.

As used herein, the term “charge density” generally refers to the difference per unit volume between the positive charge of its nucleus in the system and the distribution of the negative charges carried by the electrons in the system.

As used herein, the term “spectroscopic properties” generally refers to the properties that are characterized by spectroscopic methods such as X-ray, infrared (IR), Raman, nuclear magnetic resonance (NMR), and electronic absorption/emission spectra.

As used herein, the term “binding affinity” generally refers to the strength of the binding interaction between a single molecule (e.g. protein or DNA) to its ligand/binding partner (e.g. drug or inhibitor).

As used herein, the term “equilibrium geometry” generally refers to the molecular geometry that corresponds to the true minimum on the respective potential energy surface.

As used herein, the term “reactivity” generally refers to the impetus for which a chemical substance undergoes a chemical reaction.

As used herein, the term “hydrophobicity” generally refers to the physical property of a molecule that is seemingly repelled from a mass of water.

As used herein, the term “synthesizability” generally refers to the ability to be synthesized by a certain synthetic route.

As used herein, the term “conformational entropy” generally refers to the entropy associated with the number of conformations of a molecule.

As used herein, the term “residence time” generally refers to the amount of time for which a molecule is bound to its target molecules.

As used herein, the term “protein structure” generally refers to the three-dimensional arrangement of atoms in an amino acid-chain molecule.

As used herein, the term “protein pocket” generally refers to the cavity on the protein surface or in the interior of a protein that possesses suitable properties for binding a ligand.

As used herein, the term “protein-ligand interactions” generally refers to the interaction behavior between protein and ligand.

As used herein, the term “binding free energy” generally refers to the free energy change associated with the binding event between a molecule and its target molecules.

As used herein, the term “toxicity” generally refers to the degree to which a chemical substance or a particular mixture of substances can damage an organism.

As used herein, the term “catalyst molecules” generally refers to the substance that helps increase the rate of a chemical reaction.

As used herein, the term “conformational search” generally refers to the process of finding the energetically preferred conformations of a molecule.

As used herein, the term “barrier height” generally refers to the energy difference between the minimum and maximum on a potential energy plot.

As used herein, the term “substrate” generally refers to the chemical species being observed in a chemical reaction.

As used herein, the term “ab initio molecular dynamics” generally refers to the methodology wherein finite-temperature dynamical trajectories are generated by using forces computed on the fly from electronic structure calculations.

As used herein, the term “classical force field” generally refers to the functional form and parameter sets used to calculate the potential energy of a system of atoms or molecules or coarse-grained particles.

As used herein, the term “classical molecular dynamics” generally refers to the methodology wherein finite-temperature dynamical trajectories are generated by using forces computed on the fly from the classical force field.

The present application discloses methods and systems for studying molecule and properties thereof.

Neither the Title nor the Abstract is to be taken as limiting in any way as the scope of the disclosed invention(s). The title of the present application and headings of sections provided in the present application are for convenience only and are not to be taken as limiting the disclosure in any way.

### Machine Learning

Machine Learning (ML) can be defined as training (e.g. tuning parameters within) a flexible computer algorithm with a particular set of data. More specifically, ML includes supervised learning, semi-supervised learning, unsupervised learning, transfer learning, delta learning, and reinforcement learning. In supervised learning, parameters within an ML model are updated such that the output of the model and the true labelled data yield a similar result. In unsupervised learning, a model learns patterns within a particular dataset without labels. In semi-supervised learning, some labels are present, and others are not. In transfer learning, a model trained on a particular dataset is fine-tuned on another dataset, possibly while holding some of the parameters fixed. In delta learning, the difference between datapoints is learned rather than absolute values. These differences could be between two computational chemistry calculations, at different levels of theory, for example. In reinforcement learning, a model is used to determine what actions to take given a particular environment. A particular set of machine learning models are neural networks, which may include the layers: fully connected, convolution, pooling, skip connections etc. When many layers are connected in a neural network, this is referred to as a deep learning model. Deep learning models have many parameters and require many datapoints to reduce the error in their predictions.

### NISQ-Noisy Intermediate-Scale Quantum Technology

Any type of quantum computers may be suitable for the technologies disclosed herein. In accordance with the description herein, suitable quantum computers may include, by way of non-limiting examples: superconducting quantum computers (qubits implemented as small superconducting circuits—Josephson junctions) (Clarke, John, and Frank K. Wilhelm. “Superconducting quantum bits.” Nature 453.7198 (2008): 1031); trapped ion quantum computers (qubits implemented as states of trapped ions) (Kielpinski, David, Chris Monroe, and David J. Wineland. “Architecture for a large-scale ion-trap quantum computer.” Nature 417.6890 (2002): 709.); optical lattice quantum computers (qubits implemented as states of neutral atoms trapped in an optical lattice) (Deutsch, Ivan H., Gavin K. Brennen, and Poul S. Jessen. “Quantum computing with neutral atoms in an optical lattice.” arXiv preprint quant-ph/0003022 (2000)); spin-based quantum dot computers (qubits implemented as the spin states of trapped electrons) (Imamog, A., David D. Awschalom, Guido Burkard, David P. DiVincenzo, Daniel Loss, M. Sherwin, and A. Small. “Quantum information processing using quantum dot spins and cavity QED.” arXiv preprint quant-ph/9904096 (1999)); spatial based quantum dot computers (qubits implemented as electron positions in a double quantum dot) (Fedichkin, Leonid, Maxim Yanchenko, and K. A. Valiev. “Novel coherent quantum bit using spatial quantization levels in semiconductor quantum dot.” arXiv preprint quant-ph/0006097 (2000)); coupled quantum wires (qubits implemented as pairs of quantum wires coupled by quantum point contact) (Bertoni, A., Paolo Bordone, Rossella Brunetti, Carlo Jacoboni, and S. Reggiani. “Quantum logic gates based on coherent electron transport in quantum wires.” Physical Review Letters 84, no. 25 (2000): 5912.); nuclear magnetic resonance quantum computers (qubits implemented as nuclear spins and probed by radio waves) (Cory, David G., Mark D. Price, and Timothy F. Havel. “Nuclear magnetic resonance spectroscopy: An experimentally accessible paradigm for quantum computing.” arXiv preprint quant-ph/9709001(1997)); solid-state NMR Kane quantum computers (qubits implemented as the nuclear spin states of phosphorus donors in silicon) (Kane, Bruce E. “A silicon-based nuclear spin quantum computer.” nature 393, no. 6681 (1998): 133.); electrons-on-helium quantum computers (qubits implemented as electron spins) (Lyon, Stephen Aplin. “Spin-based quantum computing using electrons on liquid helium.” arXiv preprint condmat/0301581 (2006)); cavity quantum electrodynamics-based quantum computers (qubits implemented as states of trapped atoms coupled to high-finesse cavities) (Burell, Zachary. “An Introduction to Quantum Computing using Cavity QED concepts.” arXiv preprint arXiv: 1210.6512 (2012).); molecular magnet-based quantum computers (qubits implemented as spin states) (Leuenberger, Michael N., and Daniel Loss. “Quantum computing in molecular magnets.” arXiv preprint cond-mat/0011415 (2001)); fullerene-based ESR quantum computers (qubits implemented as electronic spins of atoms or molecules encased in fullerenes) (Harneit, Wolfgang. “Quantum Computing with Endohedral Fullerenes.” arXiv preprint arXiv: 1708.09298 (2017).); linear optical quantum computers (qubits implemented as processing states of different modes of light through linear optical elements such as mirrors, beam splitters and phase shifters) (Knill, E., R. Laflamme, and G. Milburn. “Efficient linear optics quantum computation.” arXiv preprint quant-ph/0006088 (2000).); diamond-based quantum computers (qubits implemented as electronic or nuclear spins of nitrogen-vacancy centres in diamond) (Nizovtsev, A. P., S. Ya Kilin, F. Jelezko, T. Gaebal, Iulian Popa, A. Gruber, and Jorg Wrachtrup. “A quantum computer based on NV centers in diamond: optically detected nutations of single electron and nuclear spins.” Optics and spectroscopy 99, no. 2 (2005): 233-244.); Bose-Einstein condensate-based quantum computers (qubits implemented as two-component BECs) (Byrnes, Tim, Kai Wen, and Yoshihisa Yamamoto. “Macroscopic quantum computation using Bose-Einstein condensates.” arXiv preprint quantum-ph/1103.5512 (2011)); transistor-based quantum computers (qubits implemented as semiconductors coupled to nanophotonic cavities) (Sun, Shuo, Hyochul Kim, Zhouchen Luo, Glenn S. Solomon, and Edo Waks. “A single-photon switch and transistor enabled by a solid-state quantum memory.” arXiv preprint quant-ph/1805.01964 (2018)); rare-earth-metal-ion-doped inorganic crystal-based quantum computers (qubits implemented as atomic ground state hyperfine levels in rare-earth-ion-doped inorganic crystals) (Ohlsson, Nicklas, R. Krishna Mohan, and Stefan Kröll. “Quantum computer hardware based on rare-earth-ion-doped inorganic crystals.” Optics communications 201, no. 1-3 (2002): 71-77.); metal-like carbon nanospheres based quantum computers (qubits implemented as electron spins in conducting carbon nanospheres) (Náfrádi, Bálint, Mohammad Choucair, Klaus-Peter Dinse, and László Forró. “Room temperature manipulation of long lifetime spins in metallic-like carbon nanospheres.” arXiv preprint cond-mat/1611.07690 (2016)); and D-Wave's quantum annealers (qubits implemented as superconducting logic elements) (Johnson, Mark W., Mohammad H S Amin, Suzanne Gildert, Trevor Lanting, Firas Hamze, Neil Dickson, R. Harris et al. “Quantum annealing with manufactured spins.” Nature 473, no. 7346 (2011): 194-198.), each of which is incorporated herein by reference in its entirety. In some cases, the quantum device is connected to the Internet such that it accesses the World Wide Web. In some cases, the quantum device is connected to a cloud computing infrastructure. In some cases, the quantum device is connected to an intranet. In some cases, the quantum device is connected to a data storage device.

### Quantum Annealer

A quantum annealer is an example of quantum mechanical system that may consist of a plurality of qubits.

Each qubit is inductively coupled to a source of bias called a local field bias. In some cases, a bias source is an electromagnetic device used to thread a magnetic flux through the qubit to provide control of the state of the qubit (e.g., U.S. Patent Application No. 2006/0225165, which is incorporated herein by reference in its entirety).

The local field biases on the qubits may be programmable and controllable. In some cases, a qubit control system comprising a digital processing unit is connected to the system of qubits and is capable of programming and tuning the local field biases on the qubits.

A quantum annealer may furthermore comprise a plurality of couplings between a plurality of pairs of the plurality of qubits. In some cases, a coupling between two qubits is a device in proximity to both qubits and threading a magnetic flux to both qubits. In some cases, a coupling may comprise a superconducting circuit interrupted by a compound Josephson junction. A magnetic flux may thread the compound Josephson junction and consequently thread a magnetic flux on both qubits (e.g., U.S. Patent Application No. 2006/0225165, which is incorporated herein by reference in its entirety). The strength of this magnetic flux may contribute quadratically to the energies of the quantum Ising model with the transverse field. In some cases, the coupling strength is enforced by tuning the coupling device in proximity of both qubits.

The coupling strengths may be controllable and programmable. In some cases, a quantum annealer control system comprising a digital processing unit may be connected to the plurality of couplings. In some cases, a quantum annealer control system comprising a digital processing unit may be capable of programming the coupling strengths of the quantum annealer.

In some cases, the quantum annealer performs a transformation of the quantum Ising model with the transverse field from an initial setup to a final one. In some cases, the initial and final setups of the quantum Ising model with the transverse field provide quantum systems described by their corresponding initial and final Hamiltonians.

In some cases, quantum annealers may be used as heuristic optimizers of their energy function. An example of such an analog processor is described in McGeoch, Catherine C. and Cong Wang, (2013), “Experimental Evaluation of an Adiabatic Quantum System for Combinatorial Optimization” Computing Frontiers,” May 14 16, 2013 and also disclosed in the Patent Application US 2006/0225165, each of which is incorporated herein by reference in its entirety.

In some cases, quantum annealers may be further used to provide samples from the Boltzmann distribution of a corresponding Ising model in a finite temperature. For example, Bian, Z., Chudak, F., Macready, W. G. and Rose, G. (2010), “The Ising model: teaching an old problem new tricks”, and also Amin, M. H., Andriyash, E., Rolfe, J., Kulchytskyy, B., and Melko, R. (2016), “Quantum Boltzmann Machine” arXiv:1601.02036, which is incorporated herein by reference in its entirety. This method of sampling is called quantum sampling.

In some cases, the quantum annealer is connected to the Internet such that it accesses the World Wide Web. In some cases, the quantum annealer is connected to a cloud computing infrastructure. In some cases, the quantum annealer is connected to an intranet. In some cases, the quantum annealer is connected to a data storage device.

### High-Performance Computing Device

An HPC may comprise one or more of a central processing unit (CPU), a graphics processing unit (GPU), a tensor processing unit (TPU), a field-programmable gate array (FPGA), an application-specific integrated circuit (ASIC), and a tensor streaming processor (TSP). Any other suitable processing unit that is capable of performing matrix multiplication may be used. Certain computing devices may be more efficient at operations such as matrix multiplication. These computing devices may provide additional efficiency gains over other computing systems. For example, a matrix multiplication device may be a GPU. A GPU may be a specialized electronic circuit optimized for high throughput, which can perform the same set of operations in parallel on many data blocks at a time. For example, a matrix multiplication device may be a TPU. A TPU may be a type of ASIC developed for low bit precision processing by Google Inc.; see Patent Application US 2016/0342891A1, the disclosure of which is incorporated by reference in its entirety. For example, a matrix multiplication device may be an FPGA. An FPGA may be an integrated circuit chip that comprises configurable logic blocks and programmable interconnects. It can be programmed after manufacturing to execute custom algorithms. For example, a matrix multiplication device may be an ASIC. An ASIC may be an integrated circuit chip that is customized to run a specific algorithm. In some case, an ASIC cannot be programmed after manufacturing. For example, a matrix multiplication device may be a TSP. A TSP may be a domain-specific programmable integrated chip that is designed for linear algebra computations as they may be performed in artificial intelligence applications, an example of which may be found in Gwennap, Linley, “Groq rocks neural networks,” which is incorporated entirely herein by reference for all purposes. In some cases, the high-performance computing device is connected to the Internet such that it accesses the World Wide Web. In some cases, the high-performance computing device is connected to a cloud computing infrastructure. In some cases, the high-performance computing device is connected to an intranet. In some cases, the high-performance computing device is connected to a data storage device.

Coherent Ising machine

A photonic computing device may be a coherent Ising machine (CIM). A CIM is a photonic device a powerful optimization system due to its increased, e.g., all-to-all, connectivity among spins. The coherent Ising machine may be adapted to solve the Ising problem; however, its optical pulses may instead be used to represent continuous variables. Such a modification may be also referred to as a coherent optical network.

The coherent optical network may be of various types such as any type described herein. In some cases, the coherent optical network comprises optical computing devices. In some cases, the coherent optical network comprises any or some of optical instruments such as beam splitters, free-space lasers that navigate on optical tables, fibre-ring cavities comprising optical fibres and fibre optics instruments. In some cases, the coherent optical network comprises integrated photonics.

In some cases, the coherent Ising machine computer is connected to the Internet such that it accesses the World Wide Web. In some cases, the coherent Ising machine computer is connected to a cloud computing infrastructure. In some cases, the coherent Ising machine computer is connected to an intranet. In some cases, the coherent Ising machine computer is connected to a data storage device.

### Optical Computing Devices

In some cases, the optical device comprises a network of optical parametric oscillators (OPOs) as disclosed in US Patent Application No2016/0162798 and in International Application WO2015006494 A1, the disclosure of each of which is incorporated by reference in their entirety.

In some cases, each spin of the Ising model is simulated by an optical parametric oscillator (OPO) operating at degeneracy.

Degenerate optical parametric oscillators (OPO) are open dissipative systems that experience second-order phase transition at the oscillation threshold. Because of the phase-sensitive amplification, a degenerate OPO may oscillate with a phase of either 0 or π with respect to the pump phase for amplitudes above the threshold. The phase is random, affected by the quantum noise associated with the optical parametric down conversion during the oscillation build-up. Therefore, a degenerate OPO naturally represents a binary digit specified by its output phase. Based on this property, a degenerate OPO system may be used as a physical representative of an Ising spin system. The phase of each OPO is identified as an Ising spin, with its amplitude and phase determined by the strength and the sign of the Ising coupling between relevant spins.

When pumped by a strong source, a degenerate optical parametric oscillator (OPO) takes one of two phase states corresponding to spin +1 or −1 in the Ising model. A network of N substantially identical OPO with mutual coupling are pumped with the same source to simulate an Ising spin system. After a transient period from the introduction of the pump, the network of OPOs approaches a steady state.

In some cases, wherein the amplitudes are above the threshold but a degenerate optical parametric oscillator is pumped by a weak source, it may represent a continuous variable instead of the binary spin.

The phase state selection process depends on the vacuum fluctuations and mutual coupling of the optical parametric oscillators (OPO). In some cases, the pump is pulsed at a constant amplitude, in other implementations the pump output is gradually increased, and in yet further implementations, the pump is controlled in other ways.

In some examples of an optical device, the plurality of couplings of the Ising model are simulated by a plurality of configurable couplings used for coupling the optical fields between optical parametric oscillators (OPO). The configurable couplings may be configured to be off or configured to be on. Turning the couplings on and off may be performed gradually or abruptly. When configured to be on, the configuration may provide any phase or amplitude depending on the coupling strengths of the Ising model.

Each optical parametric oscillator (OPO) output may be interfered with a phase reference and the result is captured at a photodetector. The OPO outputs represent a configuration of the Ising model. For example, a zero phase may represent a spin −1 state, and a π phase may represent a+1 spin state in the Ising model.

For the Ising model with spins, and according to some examples, a resonant cavity of the plurality of optical parametric oscillators (OPO) is configured to have a round-trip time equal to times the period of pulses from a pump source. Round-trip time as used herein indicates the time for light to propagate along one pass of a described recursive path. The pulses of a pulse train with period equal to the period of the resonator cavity round-trip time may propagate through the OPOs concurrently without interfering with each other.

In some cases, the couplings of the optical parametric oscillators (OPO) are provided by a plurality of delay lines allocated along the resonator cavity.

The plurality of delay lines comprises a plurality of modulators which synchronously control the strengths and phases of couplings, allowing for programming of the optical device to simulate the Ising model.

In a network of optical parametric oscillators (OPO), delay lines and corresponding modulators are enough to control amplitude and phase of coupling between every two OPOs

In some cases, a device capable of sampling from an Ising model can be manufactured as network of optical parametric oscillators (OPO) as disclosed in US Patent Application No. 2016/0162798, the disclosure of which is incorporated by reference in its entirety.

In some cases, the network of optical parametric oscillators (OPO) and couplings of OPOs are achieved using commercially available mode locked lasers and optical elements, such as telecom fiber delay lines, modulators, and other optical devices. Alternatively, the network of OPOs and couplings of OPOs are implemented using optical fiber technologies, such as fiber technologies developed for telecommunications applications. In some cases, the couplings may be realized with fibers and controlled by optical Kerr shutters.

In some cases, the optical computing device is connected to the Internet such that it accesses the World Wide Web. In some cases, the optical computing device is connected to a cloud computing infrastructure. In some cases, the optical computing device is connected to an intranet. In some cases, the optical computing device is connected to a data storage device.

### Integrated Photonic Coherent Ising Machine

An integrated photonic coherent Ising machine disclosed for instance in “Coherent Ising machines with error correction feedback” by Satoshi Kako, Timothée Leleu, Yoshitaka Inui, Farad Khoyratee, Sam Reifenstein, and Yoshihisa Yamamoto, the disclosure of which is incorporated by reference in its entirety is a combination of nodes and a connection network solving a particular Ising problem. In some cases, the combination of nodes and the connection network may form an optical computer that is adiabatic. In other words, the combination of the nodes and the connection network may non-deterministically solve an Ising problem when the values stored in the nodes reach a steady state to minimize the energy of the nodes and the connection network. Values stored in the nodes at the minimum energy level may be associated with values that solve a particular Ising problem.

In some cases, a system comprises a plurality of ring resonator photonic nodes, wherein each one of the plurality of ring resonator photonic nodes stores a value; a pump coupled to each one of the plurality of ring resonator photonic nodes via a pump waveguide for providing energy to each one of the plurality of ring resonator photonic nodes; and a connection network comprising a plurality of two by two building block of elements, wherein each element of the two by two building block comprises a plurality of phase shifters for tuning the connection network with parameters associated with encoding of an Ising problem, wherein the connection network processes the value stored in the each one of the plurality of ring resonator photonic nodes, wherein the Ising problem is solved by the value stored in the each one of the plurality of ring resonator photonic nodes at a minimum energy level.

In some cases, the integrated photonic coherent Ising machine computer is connected to the Internet such that it accesses the World Wide Web. In some cases, the integrated photonic coherent Ising machine computer is connected to a cloud computing infrastructure. In some cases, the integrated photonic coherent Ising machine computer is connected to an intranet. In some cases, the integrated photonic coherent Ising machine computer is connected to a data storage device.

### Digital Computer

In some cases, the digital computer comprises one or more hardware central processing units (CPUs) that carry out the digital computer's functions. In some cases, the digital computer further comprises an operating system configured to perform executable instructions. In some cases, the digital computer is connected to a computer network. In some cases, the digital computer is connected to the Internet such that it accesses the World Wide Web. In some cases, the digital computer is connected to a cloud computing infrastructure. In some cases, the digital computer is connected to an intranet. In some cases, the digital computer is connected to a data storage device.

Various types of digital computer may be used. In fact, suitable digital computers may include, by way of non-limiting examples, server computers, desktop computers, laptop computers, notebook computers, sub-notebook computers, netbook computers, set-top computers, media streaming devices, handheld computers, Internet appliances, mobile smartphones, tablet computers, personal digital assistants, video game consoles, and vehicles. Smartphones may be suitable for use with one or more examples of the method and the system described herein. Select televisions, video players, and digital music players, in some cases with computer network connectivity, may be suitable for use in some cases of the system and the method described herein. Suitable tablet computers may include those with booklet, slate, and convertible configurations.

In some cases, the digital computer comprises an operating system configured to perform executable instructions. The operating system may be, for example, software, comprising programs and data, which manages the device's hardware and provides services for execution of applications. Various types of operating system may be used. For example, suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Suitable personal computer operating systems may include, by way of non-limiting examples, Microsoft® Windows®, Apple® Mac OS X®, UNIX®, and UNIX-like operating systems such as GNU/Linux®. In some cases, the operating system is provided by cloud computing. Suitable mobile smart phone operating systems may include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Suitable media streaming device operating systems may include, by way of non-limiting examples, Apple TV®, Roku®, Boxee®, Google TV®, Google Chromecast®, Amazon Fire®, and Samsung® HomeSync®. Suitable video game console operating systems may include, by way of non-limiting examples, Sony® PS3®, Sony® PS4®, Microsoft® Xbox 360®, Microsoft® Xbox One®, Nintendo® Wii®, Nintendo® Wii U®, and Ouya®.

In some cases, the digital computer comprises a storage and/or memory device. Various types of storage and/or memory may be used in the digital computer. In some cases, the storage and/or memory device comprises one or more physical apparatuses used to store data or programs on a temporary or permanent basis. In some cases, the device comprises a volatile memory and requires power to maintain stored information. In some cases, the device comprises non-volatile memory and retains stored information when the digital computer is not powered. In some cases, the non-volatile memory comprises a flash memory. In some cases, the non-volatile memory comprises a dynamic random-access memory (DRAM). In some cases, the non-volatile memory comprises a ferroelectric random access memory (FRAM). In some cases, the non-volatile memory comprises a phase-change random access memory (PRAM). In some cases, the device comprises a storage device including, by way of non-limiting examples, CD-ROMs, DVDs, flash memory devices, magnetic disk drives, magnetic tapes drives, optical disk drives, and cloud computing based storage. In some cases, the storage and/or memory device comprises a combination of devices, such as those disclosed herein.

In some cases, the digital computer comprises a display used for providing visual information to a user. Various types of display may be used. In some cases, the display comprises a cathode ray tube (CRT). In some cases, the display comprises a liquid crystal display (LCD). In some cases, the display comprises a thin film transistor liquid crystal display (TFT-LCD). In some cases, the display comprises an organic light-emitting diode (OLED) display. In some cases, an OLED display comprises a passive-matrix OLED (PMOLED) or active-matrix OLED (AMOLED) display. In some cases, the display comprises a plasma display. In some cases, the display comprises a video projector. In some cases, the display comprises a combination of devices, such as those disclosed herein.

In some cases, the digital computer comprises an input device to receive information from a user. Various types of input devices may be used. In some cases, the input device comprises a keyboard. In some cases, the input device comprises a pointing device including, by way of non-limiting examples, a mouse, trackball, trackpad, joystick, game controller, or stylus. In some cases, the input device comprises a touch screen or a multi-touch screen. In some cases, the input device comprises a microphone to capture voice or other sound input. In some cases, the input device comprises a video camera or other sensor to capture motion or visual input. In some cases, the input device comprises a Kinect™, Leap Motion™, or the like. In some cases, the input device comprises a combination of devices, such as those disclosed herein.

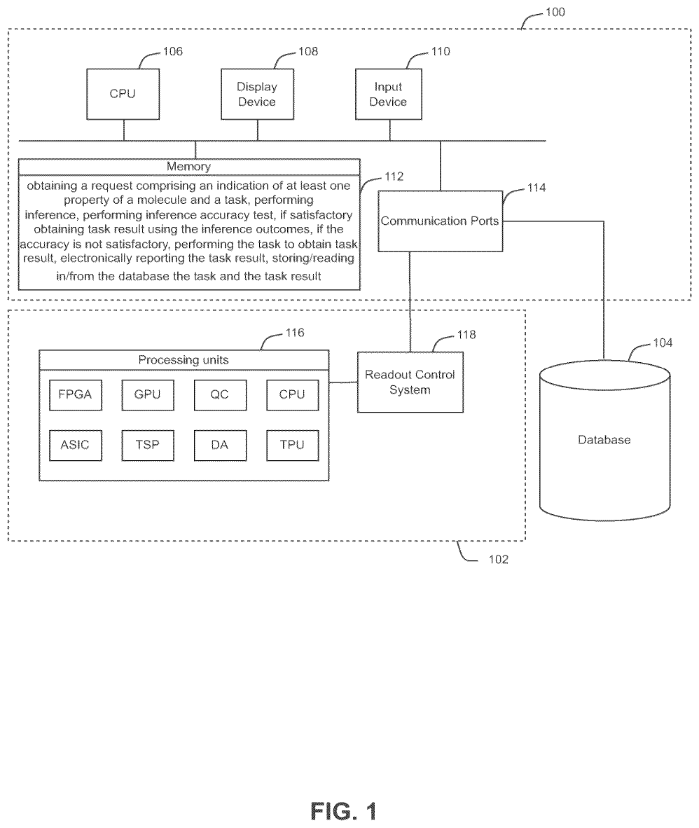

Now referring to FIG. 1, there is shown an example schematic diagram of a system for studying molecule and properties thereof. The system is configured to utilize a machine learning (ML) model.

The system may comprise a digital computer 100. The digital computer may be a digital computer of various types, such as, for example, a digital computer as described elsewhere herein. The digital computer may comprise at least one processing device 106 and at least one memory 112. The at least one memory may comprise a computer program executable by the processing device 106 which may be configured to obtain a request comprising an indication of at least one property of a molecule and a task, to perform inference on at least one machine learning (ML) model using the obtained indication, to perform inference reliability test, if the reliability is satisfactory, to obtain task result using the inference outcomes, if the reliability is not satisfactory, to perform the obtained task to obtain task result, to report electronically the task result.

The system may comprise a computational platform 102 operatively connected to the digital computer 100. The computational platform 102 may comprise at least one processor 116. The at least one processor 116 may be of various types of processors such as, for example, the types of processors as described elsewhere herein. The at least one processor can include Noisy Intermediate-Scale Quantum (NISQ) technology, any quantum device, any high-performance computing device, any quantum annealer, any optical computing device, an integrated photonic coherent Ising machine such as those disclosed elsewhere herein. For example, the at least one processor can comprise at least one field-programmable gate array (FPGA), application-specific integrated circuit (ASIC), central processing unit (CPU), graphics processing unit (GPU), tensor processing unit (TPU), tensor streaming processor (TSP), quantum computer, quantum annealer, integrated photonic coherent Ising machine, optical quantum computer, or the like, or any combination thereof. The computational platform 102 may be provided by a cloud computing platform.

Each component of the system (e.g., the hardware) may be used as part of the system to execute a whole method, or any portion thereof, alone or in combination with other components (e.g., other hardware).

In some cases, the components may be used for obtaining a request comprising an indication of at least one property of a molecule and a task, performing inference on at least one machine learning (ML) model using the obtained indication, performing inference reliability test, if the reliability is satisfactory obtaining task result using the inference outcomes, if the reliability is not satisfactory, performing the task to obtain task result.

The computational platform 102 may be operatively connected to the digital computer 100. The computational platform may comprise a read-out control system 118. The read-out control system may be configured to read information (e.g., computational results, parameters, etc.) from the at least one processor 116. For example, the read-out control system can be configured to convert data from an FPGA to data usable by a digital computer.

The system may comprise a database 104. The database 104 may be operatively connected to the digital computer 100. The database 104 may be a database of various types. The database 104 may refer to a central repository configured to save the specification of the task and task results. In some cases, the database can be, for example, MongoDB. The database 104 may be used to store indications of properties of molecules, corresponding tasks and results thereof. The database 104 may be used to store the task results. The database 104 may be further used to store the output from chemistry discovery toolkit. The database 104 may be further used to store the dataset for training the ML models. The dataset for training ML models may be a subset or complete set of task results. The dataset for training ML models may further be a subset or complete set of the output from chemistry discovery toolkit. The processing device 106 may be further configured to store in the database 104 indications of properties of molecules, corresponding tasks and the results thereof and to read from the database 104 indications of properties of molecules.

The computational platform 102 and the database 104 may be connected to the digital computer 100 over a network. The computational platform, the database, and/or the digital computer can have network communication devices. The network communication devices can enable the computational platform, the database, and/or the digital computer to communicate with each other and with any number of user devices, over a network. The network can be a wired or wireless network. For example, the network can be a fiber optic network, Ethernet® network, a satellite network, a cellular network, a Wi-Fi® network, a Bluetooth® network, or the like. In one or more implementations, the computational platform, the database, and/or digital computer can be several distributed computational platforms, databases, and/or the digital computers that are accessible through the Internet. Such computational platforms, databases, and/or digital computers may be considered cloud computing devices. In some cases, the one or more processors of the at least one processor may be located in the cloud.

The at least one processor 116 may comprise one or more virtual machines. The one or more virtual machines may be one or more emulations of one or more computer systems. The virtual machines may be process virtual machines (e.g., virtual machines configured to implement a process in a platform-independent environment). The virtual machines may be systems virtual machines (e.g., virtual machines configured to execute an operating system and related programs). The virtual machine may be configured to emulate a different architecture from the at least one processor. For example, the virtual machine may be configured to emulate a quantum computing architecture on a silicon computer chip. Examples of virtual machines may include, but are not limited to, VMware®, VirtualBox®, Parallels®, QEMU®, Citrix® Hypervisor, Microsoft® Hyper-V®, or the like.

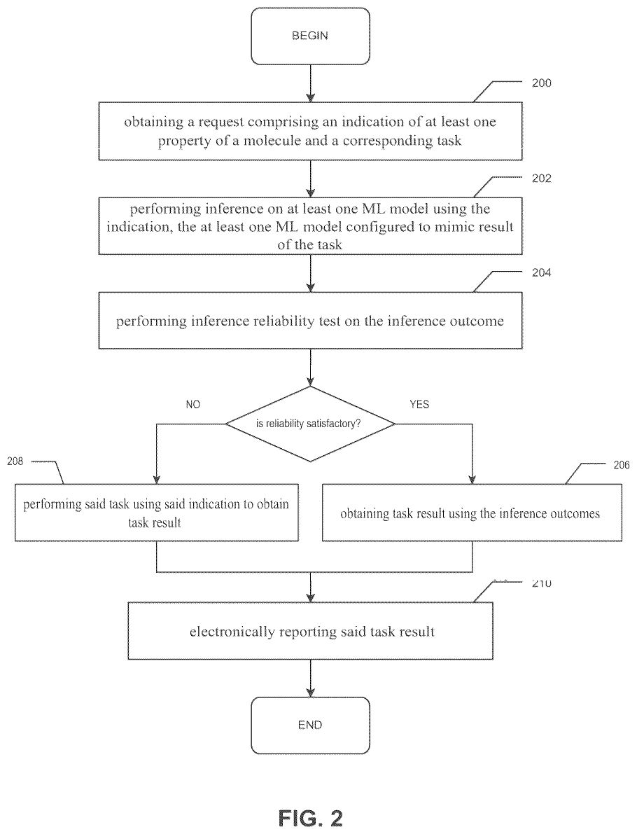

Now referring to FIG. 2, there is shown a flowchart an embodiment of a method for studying molecule and properties thereof.

According to processing operation 200, a request comprising an indication of at least one property of a molecule and a corresponding task is obtained. The property of a molecule may be of various types such as any property described elsewhere herein. In one or more embodiments, the property of a molecule may include three dimensional coordinates of the atoms on the molecule, a fingerprint, simplified molecular-input line-entry system (SMILES), or International Chemical Identifier (InChI).

In one or more embodiments, the property of a molecule may further include ground state energy, excited states energies, highest occupied molecular orbital (HOMO)-lowest unoccupied molecular orbital (LUMO) gap, ionization potential, electron affinity, singlet-triplet gap, atomic charge, dipole moment, charge density, spectroscopic properties, peak position at X nm wherein X is the peak position, and binding affinity with a target molecule, equilibrium geometry, transition state geometry, reactivity, hydrophobicity, synthesizability, conformational entropy, and residence time of a molecule interacting with another molecule, In one or more embodiments, the property of a molecule may further include effective carrier mass, acoustic wave propagation and elastic constants, the band structure, density of states, and forces on each atom and the stress tensor. In one or more embodiments, the property of a molecule may further include intercalation voltages, voltage profile, and phase diagram. In one or more embodiments, the property of a molecule may further include radial distribution functions, diffusion constant, viscosity, and conductivity.

The corresponding task may be of various types, such as any task disclosed elsewhere herein. In one or more embodiments, the task may include at least one computational chemical approach to compute at least one property of a molecule disclosed elsewhere here. In one or more embodiments, the task may include the prediction of the molecules with desired properties. In one or more embodiments, the task includes at least one member of the group consisting of protein structure prediction, protein pocket identification, virtual screening based on protein-ligand interactions, quantitative structure-activity relationship prediction, molecular similarity search, classical force field generation, classical molecular dynamics simulations, binding free energy calculations, toxicity prediction, synthesizability prediction, generation of candidate catalyst molecules, conformational search, virtual screening based on the outcome prediction and the barrier height, prediction for the reaction between catalyst candidate and substrate, ab initio molecular dynamics simulations, reaction barrier height calculation, molecular coordinate calculation, ground state energy calculation, excited states energies, HOMO-LUMO gap calculation, ionization potential calculation, electron affinity calculation, singlet-triplet gap calculation, atomic charge calculation, dipole moment calculation, charge density calculation, spectroscopic properties prediction, equilibrium geometry prediction, reactivity prediction, hydrophobicity prediction, conformational entropy prediction, residence time of a molecule interacting with another molecule prediction.

In one or more embodiments, the task includes a pipeline consisting of a plurality of subtasks.

According to processing operation 202, inference on at least one machine learning (ML) model using the obtained indication is performed. The at least one machine learning (ML) model is configured to mimic results of the task. The at least one machine learning (ML) model may be of various types, such as any machine learning model (ML) described elsewhere herein. In one or more embodiments, the machine learning (ML) model is based on supervised learning. In one or more embodiments, the machine learning (ML) model is based on unsupervised learning. In one or more embodiments, the machine learning (ML) model is based on reinforcement learning. In one or more embodiments, the machine learning (ML) model is based on active learning. In one or more embodiments, the machine learning (ML) model is based on semi-supervised learning. In one or more embodiments, the machine learning (ML) model is based on continuous learning. In one or more embodiments, the machine learning (ML) model is based on transfer learning. In one or more embodiments, the machine learning (ML) model is based on delta learning. In one or more embodiments, modern machine learning (ML) models include an Artificial Neural Network (ANN), a Convolutional Neural Network (CNN), a Graph Neural Network (GNN), a message passing neural network, a transformer network, an autoencoder (AE), a variational autoencoder (VAE), and a Generative Adversarial Network (GAN). These methods utilize automatic differentiation and gradient descent techniques. In one or more embodiments, classical machine learning (ML) modes include a kernel ridge regressor, a random forest regressor, a gradient boosting regressor, a linear regressor, a logistic regressor, a ridge regressor, a lasso regressor, a polynomial regressor, a Bayesian regressor, an elastic net regressor, a principal component regressor, a least squares regressor, a support vector regressor. These classical ML models typically do not utilize automatic differentiation and use optimization strategies other than gradient descent. For all aforementioned models, ensemble models may be constructed by combining the predictions of 2 or more individual models.

According to processing operation 204, the inference reliability test is performed. In one or more embodiments, the inference reliability test includes standard deviation computation using the inference outcomes based on plural of ML models. In one or more embodiments, the inference reliability test includes standard error computation using the inference outcomes based on plural of ML models. In one or more embodiments, the inference reliability test includes the similarity search between the training dataset and the request to identify a rareness of the request. In some embodiments, the request may comprise an indication of at least one property of a molecule, and/or a corresponding task. For example, the request may comprise the information that is needed to compare a molecule with the rest of the training set (e.g., to identify a rareness of the request), and/or the information needed to pass into the ML model to obtain a result. In one or more embodiments, the inference reliability test includes instance selection strategies for selecting critical instances to refine the ML models. Instance selection strategies include the uncertainty sampling approach, the query by committee approach, the expected model change approach, the expected error reduction approach, and the density weighted methods. In one or more embodiments, prior to processing operation 202 accuracy requirements are obtained. In one or more embodiments, prior to processing operation 202 limit for the computational resources is obtained. The accuracy requirements and the limit for the computational resources are used to perform the inference reliability test.

According to processing operation 206, if the result of the inference reliability test is satisfactory, the task result is obtained using the inference outcomes. In one or more embodiments, the inference outcome along with the standard deviation based on plural of ML models as an indication of reliability will be electronically reported as the task result.

According to processing operation 208, if the result of the inference reliability test is not satisfactory, the task is performed using the indication of at least one property of a molecule and a corresponding task to obtain the task result. In one or more embodiments, the task may be performed by using at least one computational chemistry approach in the chemistry discovery toolkit. In one or more embodiments, the task may be performed by using at least one laboratory experimental approach in the chemistry discovery toolkit. In one or more embodiments wherein the task includes a pipeline comprising a plurality of subtasks, the task is performed by performing all the subtasks in a specific order.

In one or more embodiments, operation 208 may be performed in the chemistry discovery toolkit. In one or more embodiments, the chemistry discovery toolkit may comprise computational chemistry method, quantum chemistry method, molecular mechanics method. In one or more embodiments, the chemistry discovery toolkit may further comprise procedures for performing tasks to predict at least one property of a molecule. In one or more embodiments, the chemistry discovery toolkit may further comprise the pipeline for performing tasks that are performed in a specific order. In one or more embodiments, the task may comprise computing energy, computing electronic structure, optimizing molecular geometry, performing the transition state search, performing conformational search, performing molecular similarity search, performing classical molecular dynamics simulation, performing ab initio molecular dynamics simulation, performing protein structure prediction, performing protein binding site prediction, performing virtual screening, performing protein-ligand binding structure prediction, performing free energy perturbation, performing ligand optimization, performing catalyst optimization, performing reaction path prediction, performing synthesizability prediction, performing spectroscopic information prediction, performing reactivity prediction, performing toxicity prediction, performing the binding structure prediction between enzyme and substrate, performing the structure prediction of self-assembled nanomaterials, optimizing the composition of the material, optimizing the experimental condition.

In one or more embodiments, computing electronic structure comprises at least one member of the group consisting of Hartree-Fock (HF) method, Density Functional Theory (DFT), Coupled-Cluster Single-, Double-, and perturbative Triple-excitations (CCSD(T)), Full Configuration Interaction (FCI), Heat-Bath Configuration Interaction (HBCI), Quantum Monte Carlo Full Configuration Interaction (QMCFCI), Density Matrix Embedding Theory (DMET), Fragment Molecular Orbital method (FMO), Incremental Full Configuration Interaction (iFCI), ML-based Schrodinger equation solver such as Paulinet, Hybrid quantum mechanics-molecular mechanics (QM/MM), and Ab initio molecular dynamics (AIMD) simulation, Variational Monte Carlo, and Diffusion Monte Carlo.

In one or more embodiments, the task result includes at least one member of the group consisting of the number indicating the ground state energy, three-dimensional coordinates of the atoms in the molecule, and the numbers indicating the peak positions of a spectroscopy information.

In one or more embodiments, the obtained task result is used for training the at least one ML model to improve performance thereof. In one or more embodiments, the obtained indication of the at least one property of the molecule, the task and the task result are stored in a database. The database may be of various types such as any database disclosed herein with respect to FIG. 1. In one or more embodiments, prior to processing operation 202 if the indication of the at least one property of the molecule, the task and the task results are stored in the database the task result is read from the database and is electronically reported. In one or more embodiments, the training may be performed when the obtained task result stored in a database could lead to improvement of the ML model's performance. In one or more embodiments, the training may be performed when the number of obtained task results stored in a database becomes sufficient for the ML model's performance improvement. In one or more embodiments, the training may be performed based on the user's request. In one or more embodiments, the training may be performed on a predetermined schedule.

In one or more embodiments, the ML model trained by using at least one obtained task result may be uploaded to the system as an additional ML model in the system. In one or more embodiments, the existing ML model in the system may be replaced by the ML model trained by using at least one obtained task result.

According to processing operation 210, the task result is reported electronically. The result can be found through a user interface on a webpage, or it can be returned to the user within a terminal window on a unix system. The user can use this result to compare to the result of a traditional calculation, or compare the result to the result of other ML models. The results can be used to perform molecular dynamics, or to compute binding energies, to name a couple examples.

Performing task may comprise at least one pipeline consisting of generating a set of three-dimensional coordinates (conformations) of the molecule based on SMILES, performing the geometry optimization calculation by using one of the said quantum chemistry methods for each conformation, performing the vibrational frequency calculation by using one of the said quantum chemistry methods for each optimized geometry and select the geometries that are at local minima by checking there is no imaginary mode, and identifying the molecular conformation that has the lowest energy as the optimized geometry of the requested molecule.

Performing task may comprise at least one pipeline consisting of performing the reaction path prediction by using Nudged Elastic Band (NEB) method and identifying the optimal reaction path between the reactant and the product, identifying the geometry that has the highest energy along the reaction path as the transition state geometry, and obtaining the reaction barrier height as the energy difference between the energy of the reactant and that of the transition state geometry.

Performing task may comprise at least one pipeline consisting of performing the excited state energy calculation by using one of the said quantum chemistry methods for the molecule, estimating the oscillator strengths as a function of excited energies (the transition lines), and convoluting these lines with a Gaussian function to turn them into spectrum.

Performing task also comprises at least one pipeline consisting of generating the chemically plausible organic molecules, performing the geometry optimization for each molecule, performing the spectroscopy calculation for each molecule, and selecting the molecules that have the strong peaks at desired wavelengths, and creating a list of them.

Performing task also comprises at least one pipeline consisting of assigning classical force field parameters to each atom in the protein-ligand complex, adding water molecules and counter ions as the solvent to the protein-ligand complex, performing the geometry optimization to remove the steric crash between atoms in the system, performing the molecular dynamics simulation, measuring the simulation time for which the ligand stays in the ligand binding site of the protein and report it as the residence time.

Performing task may comprise at least one pipeline consisting of downloading the three-dimensional coordinates of the protein from the Protein Data Bank (PDB) server by the requested PDB ID, adding missing atoms and residues to the protein coordinate, performing the geometry optimization to relax the protein structure, performing the binding pocket prediction to identify the desired interaction site between the protein and the ligand, generating the three-dimensional coordinates of the ligands based on SMILES in the given ligand database, performing the binding free energy calculation between the ligand and the protein pocket for each ligand in the ligand database, sorting the ligands based on the strength of the binding free energy, and identifying the ligand that has the strongest binding free energy.

Performing task may comprise at least one experimental pipeline consisting of synthesizing Janus type motifs by adding a spacer between the complementary guanine and cytosine paris, examine the aggregation (nanotube formation) behavior for each Janus type motifs by using scanning electron microscopy (SEM), selecting the Janus type motifs that formed the nanotubes, performing the tapping mode atomic force microscopy (TM-AFM) and transmission electron microscopy (TEM) to measure the outer diameter of nanotubes, and identifying the Janus type motifs that formed the nanotube with the desired outer diameter.

What is claimed is:

1. A method for studying molecule and properties thereof, the method comprising:
a) obtaining a request comprising an indication of at least one property of a molecule and a corresponding task;
b) performing inference on at least one machine learning (ML) model using said indication, the at least one machine learning (ML) model configured to mimic result of said task to generate an inference outcome;
c) performing inference reliability test on the inference outcome;
i) obtaining task result using the inference outcome in response to a satisfactory inference reliability test;
ii) performing said task using said indication to obtain task result in response to a nonsatisfactory inference reliability test;
d) outputting said task result.

2. The method of claim 1, further comprising training the at least one ML model using a training dataset, wherein said training dataset comprises said task result obtained in c) ii) in response to the nonsatisfactory inference reliability test.

3. The method of claim 1, further comprising, storing, in response to the nonsatisfactory inference reliability test, in a database said indication of said at least one property of said molecule, said corresponding task, and said task result obtained in c) ii).

4. The method of claim 3, further comprising, prior to b), outputting a task result if said indication of said at least one property of said molecule, said corresponding task and said task results are stored in said database.

5. The method of claim 1, further comprising, prior to b), obtaining at least one of accuracy requirements and limit for computational resources.

6. The method of claim 1, wherein said indication of at least one property of a molecule comprises at least one member of the group consisting of three dimensional coordinates of the atoms on the molecule, a fingerprint, simplified molecular-input line-entry system (SMILES), International Chemical Identifier (InChI), description of the shape of the spectroscopic, and name of the property.

7. The method of claim 1, wherein said at least one property comprises at least one member of the group consisting of molecular coordinate, ground state energy, excited states energies, highest occupied molecular orbital (HOMO)-lowest unoccupied molecular orbital (LUMO) gap, lonization potential, electron affinity, singlet-triplet gap, atomic charge, dipole moment, charge density, spectroscopic properties, peak position at X nm wherein X is the peak position, and binding affinity with a target molecule, equilibrium geometry, reactivity, hydrophobicity, synthesizability, conformational entropy, and residence time of a molecule interacting with another molecule.

8. The method of claim 1, wherein said task comprises at least one member of the group consisting of protein structure prediction, protein pocket identification, virtual screening based on protein-ligand interactions, quantitative structure-activity relationship prediction, molecular similarity search, classical force field generation, classical molecular dynamics simulations, binding free energy calculations, toxicity prediction, synthesizability prediction, generation of candidate catalyst molecules, conformational search, virtual screening based on the outcome prediction and the barrier height, prediction for the reaction between catalyst candidate and substrate, ab initio molecular dynamics simulations, reaction barrier height calculation, molecular coordinate calculation, ground state energy calculation, excited states energies, HOMO-LUMO gap calculation, ionization potential calculation, electron affinity calculation, singlet-triplet gap calculation, atomic charge calculation, dipole moment calculation, charge density calculation, spectroscopic properties prediction, equilibrium geometry prediction, reactivity prediction, hydrophobicity prediction, conformational entropy prediction, residence time of a molecule interacting with another molecule prediction.

9. The method of claim 1, wherein said task comprises a pipeline comprising a plurality of subtasks.

10. The method of claim 1, wherein said task result comprises at least one member of the group consisting of the number indicating the ground state energy, three-dimensional coordinates of the atoms in the molecule, and the numbers indicating the peak positions of a spectroscopy information.

11. The method of claim 1, wherein said performing said task in c) ii) comprises at least one member of the group consisting of performing a corresponding experiment, implementing computational chemistry method, quantum chemistry method, molecular mechanics method, computing energy, computing electronic structure, optimizing molecular geometry, performing the transition state search, performing conformational search, performing molecular similarity search, performing classical molecular dynamics simulation, performing ab initio molecular dynamics simulation, performing protein structure prediction, performing protein binding site prediction, performing virtual screening, performing protein-ligand binding structure prediction, performing free energy perturbation, performing ligand optimization, performing catalyst optimization, performing reaction path prediction, performing synthesizability prediction, performing spectroscopic information prediction, performing reactivity prediction, performing toxicity prediction, performing the binding structure prediction between enzyme and substrate, performing the structure prediction of self-assembled nanomaterials, optimizing the composition of the material, optimizing the experimental condition.

12. The method of claim 11, wherein said performing structure prediction comprises using a quantum chemistry method using at least one member of the group consisting of Hartree-Fock (HF) method, Density Functional Theory (DFT), Coupled-Cluster Single-, Double-, and perturbative Triple-excitations (CCSD(T)), Full Configuration Interaction (FCI), Heat-Bath Configuration Interaction (HBCI), Quantum Monte Carlo Full Configuration Interaction (QMCFCI), Density Matrix Embedding Theory (DMET), Fragment Molecular Orbital method (FMO), Incremental Full Configuration Interaction (iFCI), ML-based Schrodinger equation solver such as Paulinet, Hybrid quantum mechanics-molecular mechanics (QM/MM), and ab initio molecular dynamics (AIMD) simulation.

13. The method of claim 1, wherein said at least one machine learning (ML) model comprises supervised learning, unsupervised learning, semi-supervised learning, reinforcement learning, active learning, delta learning, continuous learning, and transfer learning.

14. The method of claim 1, wherein said inference reliability test comprises standard deviation computation using the inference outcome.

15. The method of claim 1, wherein said inference reliability test comprises standard error computation using the inference outcome.

16. The method of claim 1, wherein said inference reliability test comprises a similarity search between the training dataset and the request to identify a rareness of the request.

17. The method of claim 1, wherein said inference reliability test comprises instance selection strategies for selecting critical instances to refine the ML models, wherein the instance selection strategies comprise an uncertainty sampling approach, a query by committee approach, an expected model change approach, an expected error reduction approach, and density weighted methods.

18. A system for studying molecule and properties thereof, the system comprising:
a) a memory component, comprising instructions for studying molecule and properties thereof and at least one trained machine learning (ML) model;
b) a computational platform operatively coupled to said memory component, wherein the computational platform is configured to execute said instructions to at least:
i) obtain a request comprising an indication of at least one property of a molecule and a corresponding task;
ii) perform inference on at least one machine learning (ML) model using said indication, the at least one machine learning (ML) model configured to mimic result of said task to generate an inference outcome;
iii) perform inference reliability test on the inference outcome; obtain task result using the inference outcomes in response to a satisfactory inference reliability test; perform said task using said indication to obtain task result in response to a nonsatisfactory inference reliability test.

19. The system of claim 18, further comprising a database configured to store tasks and results thereof, wherein said instructions comprise storing in said database said indication of said at least one property of said molecule, said task and said task result.

20. The system of claim 18, further comprising a distributed computing system.

21. The system of claim 18, further comprising a cloud computing system.

22. The system of claim 18, further comprising a classical computer.

23. The system of claim 18, further comprising a non-classical computer.

24. The system of claim 23, wherein said non-classical computer is a quantum computer.

25. The system of claim 24, wherein said quantum computer comprises at least one member of the group consisting of quantum devices, such as superconducting quantum computers, trapped ion quantum computers, optical quantum computers, or quantum annealer.
